(19) United States
(12) Patent Application Publication
(10) Pub. No.: US 2006/0292110 A1
(43) Pub. Date: Dec. 28, 2006

(54) Coffee aroma vehicle air freshener

(76) Inventor: Ramona Reinhardt, Scottsdale, AZ (US)

Correspondence Address: NATIONAL IP RIGHTS CENTER, LLC;SCOTT J. FIELDS, ESQ., 550 TOWNSHIP LINE ROAD, SUITE 400, BLUE BELL, PA, 19422, US

(21) Appl. No.: 11/474,767

(22) Filed: Jun. 26, 2006

Related U.S. Application Data
(60) Provisional application No. 60/693,975, filed on Jun. 24, 2005.

(51) Int. Cl. A61L 9/00

(52) U.S. Cl. 424/76.2

(57) ABSTRACT

A car freshener comprising of a pre-formed material constructed to absorb a scent and a coffee flavored scent affixed to the pre-formed material.

### CLAIM OF PRIORITY

This application claims the priority of U.S. Provisional Application No. 60/693,975, filed Jun. 24, 2005.

### FIELD OF THE INVENTION

The present invention is directed to a novel system for creating coffee scents.

### BACKGROUND OF THE INVENTION

Coffee is one of the most popular drinks in the world. One of the most important and alluring aspects of coffee is the aroma of coffee. Like chocolate, coffee has an extremely attractive fragrance. It would be desirable to provide a scent mechanism by which coffee aroma can be placed in a car. Many people utilize various car fresheners in order to make their vehicles smell better. Among the most popular scents are “new car” and lemon. The problem with these car fresheners is that they often add an unusual odor to the car.

There have been a number of patents directed to car fresheners. U.S. Pat. No. 5,865,372 discloses a sphere with undulations, serving as vanes, which is pivotally mounted on a stand which has means for clipping the stand to an air vent in an automobile. The sphere has molded therein a fragrance which will be time released when exposed to heat or air currents.

U.S. Pat. No. 5,373,581 discloses an automobile plug-in air freshener with a main body portion in a cylindrical configuration having an open outboard end and a closed inboard end and pluggable into an electrical cigarette lighter socket of a vehicle. An extension portion has a cone shaped interior component rotatably positioned outboardly of the open end of the cylinder with an interior edge in a cylindrical configuration positioned rotatably in the outboard end of the main body portion with an exterior component in a rectangular configuration constituting a housing with an opened inboard end and louvers at the outboard end and a slot along an upper intermediate extent. A generally planar refill container in a rectangular configuration is removably positionable within the slot of the rectangular housing, the refill container having a fragrance therein activated by heat. A heating element is located within the main body portion adjacent to the outboard open end is energized by a switch actuated by rotation of the extension portion to heat the fragrance within the refill cartridge for disseminating a fragrance and freshening the adjacent air.

U.S. Pat. No. 4,968,456 discloses an electrical air freshener automobile cigarette lighter. The air freshener includes a housing or body; an electrical motor within the housing connectable to a source of electrical current; a switch on the exterior of the housing for switching on and off the electrical current to the motor; air flow inlets at the back of the housing; odor emitting means mounted at the front of the housing; a fan connected to the motor and mounted within the housing such that operation of the motor creates air flow into the housing through the inlets, past the fan and motor and out through the odor emitting means. A stem protrudes from the back of the housing and is attached for insertion into the cigarette lighter receptacle. In the preferred embodiment the stem extends non-perpendicularly from the rear of the housing.

The above patents are not related to the use of coffee aromas or scents.

It is an object of the present invention to provide a mechanism whereby the scent of coffee can be saved and stored on a scent applicator.

It is a further object of the present invention to provide novel coffee scent applicators.

These and other objects of the invention will become apparent from the detailed description which follows.

### SUMMARY OF THE INVENTION

In a preferred embodiment, the present invention is a car freshener comprising a pre-formed material constructed to absorb a scent with a coffee flavored scent affixed to the pre-formed material.

In yet a further embodiment, the present invention comprises a pre-formed material constructed to absorb a scent which is affixed to a coffee lid and a coffee-flavored scent affixed to the pre-formed material.

In a still further embodiment, the present invention comprises a pre-formed material constructed to absorb a scent and a coffee-flavored scent affixed to the material.

In a further embodiment, the present invention comprises a coffee-scented car freshener comprising an online interface for selecting from a plurality of coffee scents divided by means for ordering a selected coffee scent.

In still a further embodiment, the invention is an air freshener comprising a perforated bag having a plurality of coffee beans therein and a card with the logo of the coffee company which manufactures the beans attached thereto.

### DESCRIPTION OF THE FIGURES

FIG. 1 is an overhead view of the present invention.

FIG. 2 is a side view of the present invention attached to a coffee cup.

FIG. 3 illustrates the present invention attached to a rear view mirror.

FIGS. 4 and 5 illustrate a web-based interface.

FIGS. 6 and 7 illustrate additional dispensing mechanisms in accordance with the present invention.

FIG. 8 is a further embodiment of the present invention.

### DETAILED DESCRIPTION OF THE INVENTION

The present invention as described with reference to the enclosed Figures wherein the same numbers are used where applicable. The present invention comprises a novel coffee aroma car and vehicle freshener.

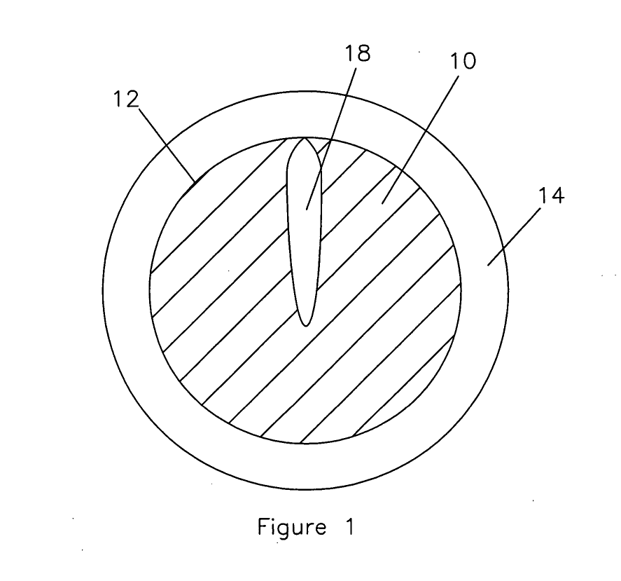

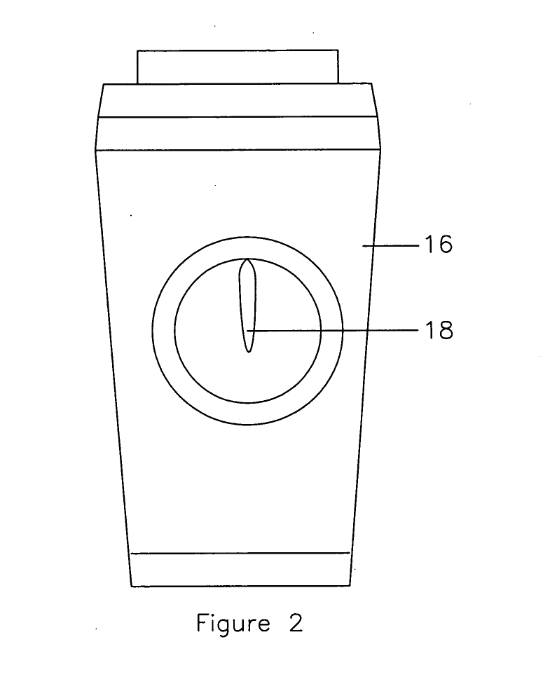

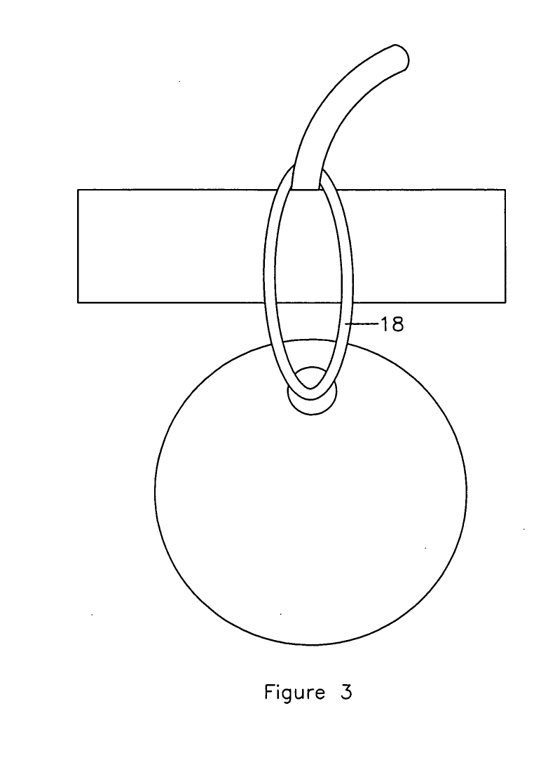

Referring to FIGS. 1 through 3, the invention comprises a coffee flavored scent applicator. The applicator is affixed to the top of or side of a coffee cup or lid. The scent applicator 10 can be disk-shaped 12 and pre-loaded on to the annular sleeve 14 on the top of a coffee cup 16. Preferably, the applicator is made from 120# blotting paper. The card can also be made from a non-woven, porous materials or synthetic carrier materials such as extruded polyethylene or molded polystyrene based materials that will hold fragrance and allow evaporative admittance of the fragrance. Such materials include, for example Tyvek®; sheeting available from E. I. duPont de Nemours & Co.; Teslin®; microporous sheeting available from PPG Industries, Inc. of Pittsburgh, Pa.; Porex®, porous plastic sheeting available from Porex Technologies Corp. of Fairburn, Ga.; or Celwa® paper pads, available from John H. Willig d/b/a Celwa Products Co. of New York, N.Y.

The coffee aromas can be obtained using a variety of well known processes including distillation and the use of synthetic solvents such as perchloroethylene and methylene chloride and fractions of petroleum such as hexane. Methylene chloride (dichloromethane) and hexane are now very widely used for the extraction of flavor and fragrance oils and pharmacologically active principles from plant materials. A more recent approach has been to employ super-critical carbon dioxide as a solvent. The present invention can also utilize non-chlorinated fluorocarbon solvents which have been developed as a replacement for CFC's such as dichlorodifluoromethane and which are presently only employed in the refrigeration industry. U.S. Pat. No. 5,512,285 discloses such a technique and is incorporated herein by reference.

The scent applicator has a small tag 18 to attach the scent applicator to the rear view mirror of a vehicle which can be removed. It has the aroma of a popular coffee. The coffee may be Hazelnut, French Vanilla, or the like. The applicator may be attached to a coffee cup or sold separately.

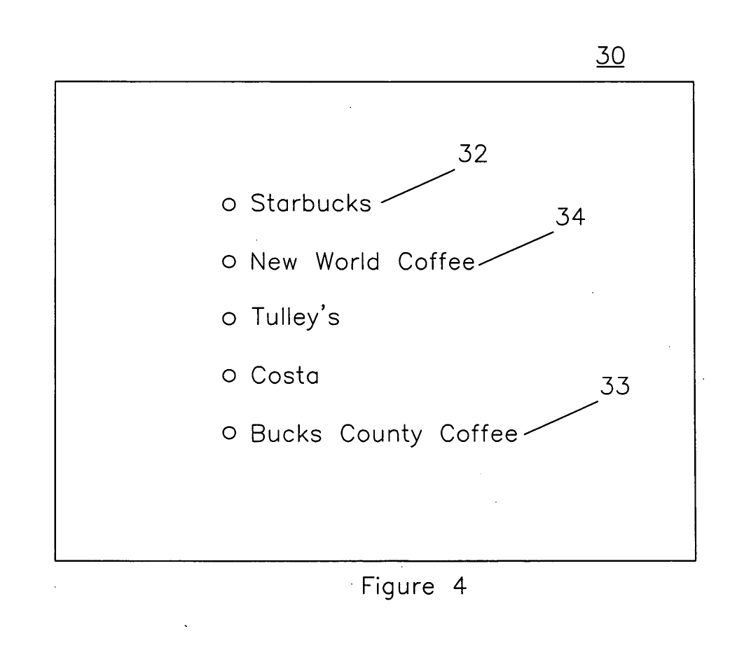

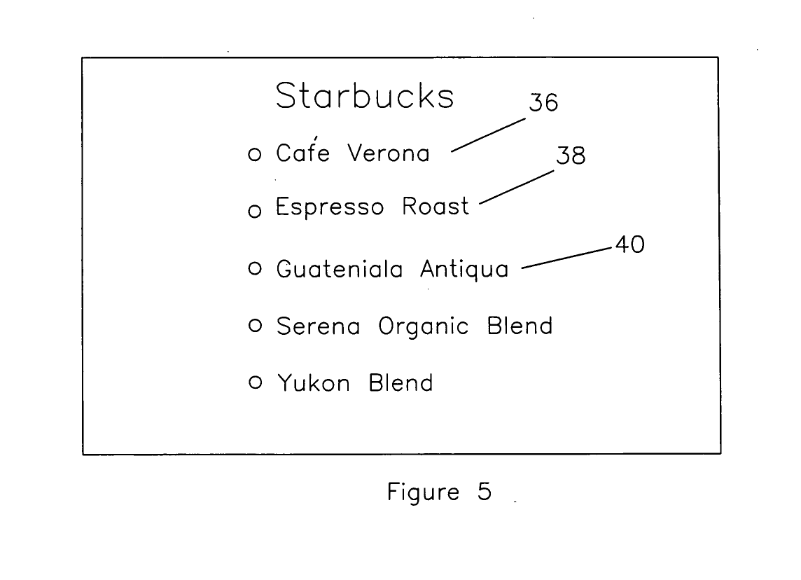

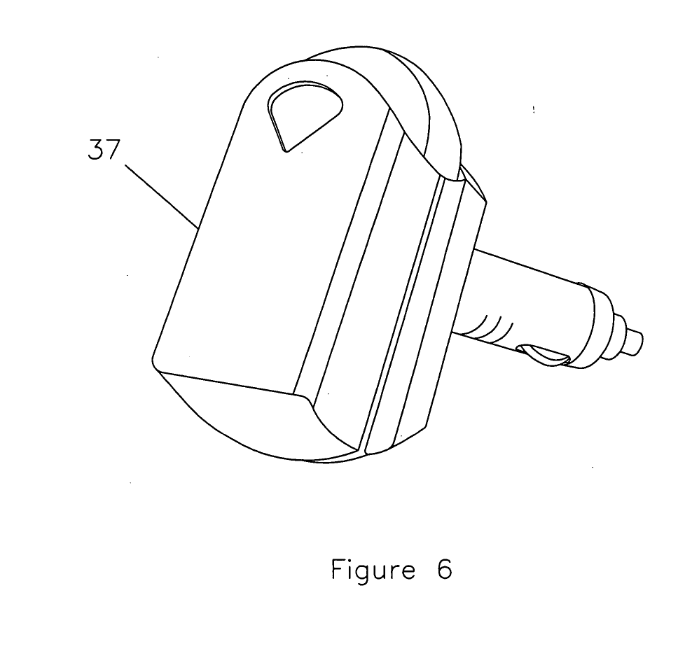

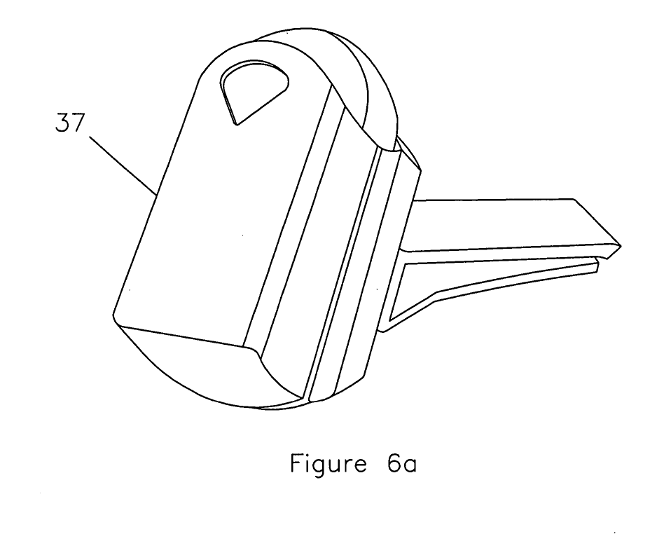

FIGS. 4 and 5 disclose an online ordering system. In an online system an individual customer can order a number of coffee scents from a variety of vendors, such as Starbucks 32, Bucks County 33 and New World Coffee 34. A specific vendor, such as Starbucks, can sell a variety of scented applicators or popular selection such as Cafe Verona 36, Espresso Roast 38 or Guanteniala Antiqua 40.

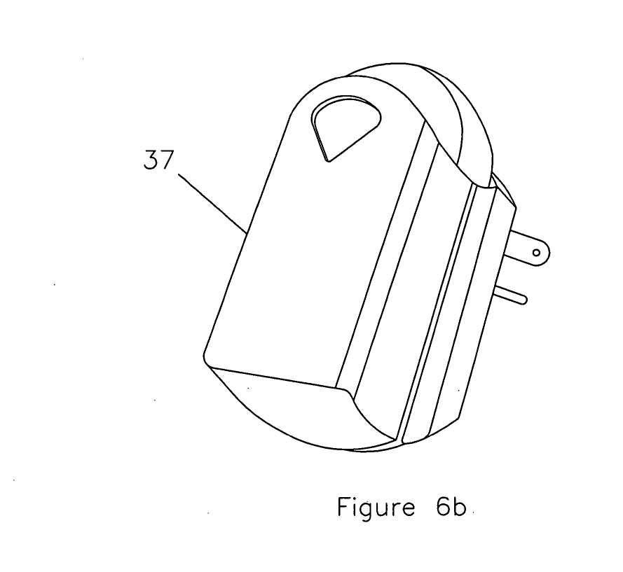

In a further embodiment, shown in FIGS. 6 and 6a, the invention comprises an insert for application into a plug in wall freshener such as a Glade plug-in like wall freshener or a similar plug-in device. In this application, the plug in wall freshener 37 will have a preloaded and/or reloadable scent cartridge of a popular coffee flavor such as Starbucks' Café Verona and/or Espresso Roast.

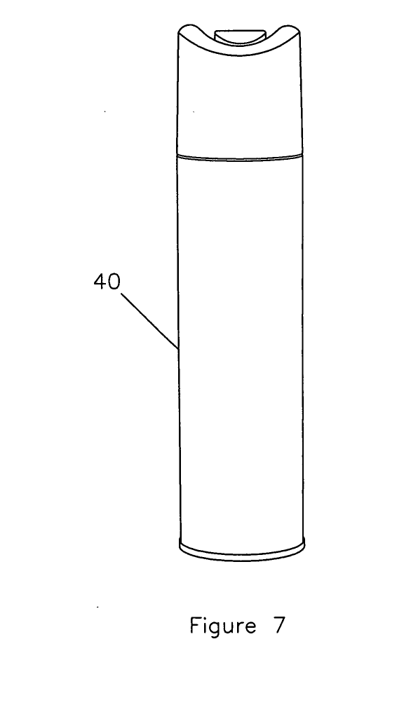

In still a further embodiment, as shown in FIG. 7, the Coffee aroma can be placed within an aerosol can 40 and then dispensed, like a commercial deodorizer (not shown).

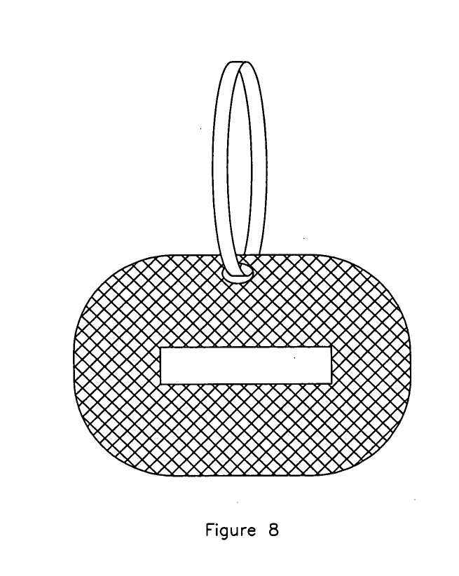

Finally, as shown in FIG. 8, the air freshener can comprise a small perforated “linen” type bag with the actual coffee beans inside. The bag can have a small card with the logo of the coffee company attached thereto.

The present invention has been described with reference to the above-discussed preferred embodiment. The true nature and scope of the invention is to be determined with reference to the attached claims.

1. A car freshener comprising:
a pre-formed material constructed to absorb a scent; and
a coffee-flavored scent affixed to the pre-formed material.

2. A car freshener comprising:
a pre-formed material constructed to absorb a scent and affixed to a coffee lid; and
a coffee-flavored scent affixed to the pre-formed material.

3. A car freshener comprising:
a pre-formed material constructed to absorb a scent; and
a coffee-flavored scent affixed to the material.

4. An air freshener comprising:
perforated bag having a plurality of coffee beans therein: and
a card with the logo of the coffee company which manufactures the beans attached thereto.

5. An on-line system for ordering a coffee-scented car freshener comprising:
an online interface for selecting from a plurality of coffee scents divided by vendor; and
means for ordering a selected coffee scent.

6. An air freshener comprising:
a perforated bag having a plurality of coffee beans therein; and
a card with the logo of the coffee company which manufactures the beans attached thereto.
